(19) United States
(12) Patent Application Publication
(10) Pub. No.: US 2021/0007979 A1
(43) Pub. Date: Jan. 14, 2021

(54) SUBLINGUAL TABLET COMPRISING SILDENAFIL CITRATE

(71) Applicant: FULTON MEDICINAL S.P.A., Arese (IT)

(72) Inventor: Enzo DE TOMMASO, Arese (IT)

(73) Assignee: FULTON MEDICINAL S.P.A., Arese (IT)

(21) Appl. No.: 16/980,158

(22) PCT Filed: Mar. 6, 2019
(86) PCT No.: PCT/EP2019/055589

§ 371 Date: Sep. 11, 2020

(30) Foreign Application Priority Data
Mar. 13, 2018 (IT) 102018000003507

(51) Int. Cl. A61K 9/00; A61K 9/20; A61K 31/519

(52) U.S. Cl. CPC A61K 9/0056; A61K 9/2013; A61K 9/2095; A61K 9/2059; A61K 31/519

(57) ABSTRACT

The invention is directed to a sublingual tablet comprising sildenafil citrate in an amount comprised between 10 mg and 150 mg, and a non-ionic surfactant in an amount comprised between 20 and 100% by weight of the amount of sildenafil citrate, wherein the nonionic surfactant is a polyethoxylated compound comprising a polar and a non-polar part, wherein the polar part of the molecule comprises from 3 to 8 ethoxy groups and the non-polar part is a C₄-C₂₀ alkyl group or a C₇-C₂₀ aryl group. The sublingual tablet is prepared by a process that comprises the following steps: introducing sildenafil citrate into a mixer; adding to the mixer containing sildenafil citrate the nonionic surfactant in at least three aliquots and mixing for 5 minutes after the addition of each aliquot. The tablet according to the invention is characterized by a high concentration of sildenafil, is easy to take, and with a good taste in the mouth.

The present invention is directed to a tablet for sublingual administration comprising sildenafil citrate and a non-ionic surfactant.

Sildenafil citrate remains the most important drug for treating erectile disfunction. Traditionally, it is orally administered; however, oral administration presents important aspects in drug development. In particular, some drawbacks, such as the necessity of taking the pill well in advance of the real need, and the possible negative impact of a heavy meal on the bioavailability of the drug. Orally administered sildenafil takes about 30 minutes before being detected in blood, and about 45 minutes to take full effect.

Individual variability in dose-effect relationship is an important factor, even when considering the dose per kg of weight of patient. Different patients respond in a very different way at the same dosage per kg of patient, sometimes with an even excessive response, and sometimes, at the same dosage, with a too weak response. To decrease this variability, sublingual administration has been chosen. In fact, sublingual administration presents the advantage of having the active substance adsorbed through the oral mucosae by the vascular plexus of the tongue, which is very reach of blood vessels. These blood vessels carry blood directly to vena cava, thus directly in the blood stream without passing through the liver.

Sublingual sildenafil citrate tablets have been proposed in the prior art. WO 2010/118516 discloses a sublingual tablet comprising 25 mg of sildenafil citrate and 30 mg of chitosan. The patent application shows that the sublingual administration leads to detection of sildenafil much more quickly than by oral administration. However, the application only shows a dosage of 25 mg sildenafil, which is normally a very low dose, being 50 to 100 mg the common dosage, and the amount of chitosan in the tablet is 120 wt % of the amount of sildenafil. Thus, chitosan is not very effective in enhancing adsorption of sildenafil through the sublingual membrane.

WO 2011/030351 discloses (example 7) sublingual sildenafil citrate tablets comprising 35 mg of sildenafil, lauroyl macrogol glycerides and other excipients. The total weight of the tablet is 375 mg. Sildenafil citrate represents therefore only 9 wt % of the tablet.

It still exists the need of a sublingual sildenafil formulation which is effective, allows a high concentration of sildenafil, is easy to take, and with a good taste in the mouth.

The present invention is directed to a sildenafil citrate composition for sublingual administration, which composition comprises sildenafil and a non-ionic surfactant in an amount comprised between 20 and 100% by weight, preferably 30 to 80% by weight of the amount of sildenafil citrate. Preferably, sildenafil citrate is present in the tablet according to the invention in an amount comprised between 10 mg and 150 mg, more preferably between 30 mg and 120 mg, even more preferably between 50 mg and 100 mg, and in term of percentage, the sildenafil citrate is present in an amount equal to or higher than 10 wt % based on the total weight of the tablet, more preferably equal to or higher than 12 wt % based on the total weight of the tablet, even more preferably equal to or higher than 14 wt % based on the total weight of the tablet.

The invention is also directed to a method for the preparation of sublingual sildenafil tablets which method comprises a step of mixing sildenafil citrate and the non-ionic surfactant.

The non-ionic surfactant is preferably a polyethoxylated compound comprising a polar and a non-polar part, wherein the polar part of the molecule comprises from 3 to 8 ethoxy groups and the non-polar part is a C₄-C_{20,} preferably a C₆-C₁₂ alkyl group, or a C₇-C_{20,} preferably a C₈-C₁₄ aryl group. A preferred nonionic surfactant is Labrasol™, which comprises a capryl or caproyl group and a C₈ ethoxy group. Preferably, the non-ionic surfactant is liquid at room temperature.

The non-ionic surfactant of the present invention plays an important role. In fact, sildenafil citrate is sparingly soluble in water, has a low permeability of the oral mucosae and undergoes metabolic degradation. It is therefore important to vehiculate Sildenafil citrate by using a compound which can help dissolving the drug and letting it through the oral mucosae membranes. The non-ionic surfactants according to the invention play this important role and increase bio availability of sildenafil citrate.

Non-ionic surfactants used in the present invention generally belong to the class of lipids. Lipids are reacted in the mouth by the enzyme lipase which forms a colloidal dispersion of lipids and increases the affinity of the drug towards the aqueous mono layer.

Without being bound to any theory, it is believed that the presence of the non-ionic surfactant according to the invention results in the formation of colloids, micelles or lamellar structures (liposomes) comprising sildenafil citrate and the non-ionic surfactant. These structures vehiculate sildenafil citrate and make it easier for the drug to pass the epithelial tissue thanks to the capacity of the non-ionic surfactant to create strong bonds with the epithelial tissue, which bonds produce opening in the membrane and allow the passage of the drug through it.

The composition according to the invention preferably comprises other ingredients such as a disintegrant, sweeteners, fragrances, binders, lubricants, excipients and other inactive ingredients.

Disintegrants are agents added to tablet formulations to promote the breakup of the tablet into smaller fragments in an aqueous environment thereby increasing the available surface area and promoting a more rapid release of the drug. Any disintegrant known in the art can be used in the present invention, e.g., disintegrants based on sodium starch glycolate (Explotab™, Primojel™, Vivastar P™), disintegrants based on mannitole (Pharmaburst™, Ludiflash™, F-Melt™), sodium bicarbonate, alginic acid, ion exchange resins. Preferred disintegrant are commercial mixtures based on sodium starch glycolate such as Explotab™. The amount of disintegrant used in the tablets according to the invention is preferably comprised between 100 and 200% by weight based on the amount of sildenafil citrate.

Other ingredients preferably present in the composition are binders, such as polyvynil pirrolidone (PVP), starch and microcrystalline cellulose (which can also help disintegration of the tablet).

In a preferred embodiment, the composition also comprises at least one lubricant, e.g. a hydrogenated castor oil, a stearate salt, talc, etc.

Preferably, the composition also comprises a desiccant compound, such as fumed silica.

The invention also relates to a process for the preparation of sildenafil sublingual tablets, which process comprises the following steps:
a. putting in a vessel an amount of sildenafil citrate;
b. adding to the vessel in at least 3 aliquots, preferably at least four aliquots, most preferably in five aliquots, an amount of nonionic surfactant, mixing after the addition of each aliquot for at least 5 minutes.

Preferably, the process further comprises the step of:
c. adding all the other ingredients one by one or in groups of two to four ingredients at the time and mixing for at least five minutes after each addition; and/or
d. feeding the mixture to a tablet press machine.

Step b) is a very important step, since the better is the incorporation of the non-ionic surfactant, preferably Labrasol™, which is an oil, into sildenafil citrate, the better will be the capability of the non-ionic surfactant to increase adsorption of sildenafil citrate through the epithelial tissue.

### EXAMPLE

Sublingual sildenafil tablets were prepared having the following composition:
Sildenafil citrate: 50.0 mg
Corn starch: 60.0 mg
Explotab™: 75.0 mg
Microcrystalline cellulose 65.0 mg
Aspartame: 10.0 mg
Menthol: 3.0 mg
PVP K25: 15.0 mg
Talc: 6.0 mg
Compritol™: 10.0 mg
Cutina HR: 15.0 mg
Aerosil 200™: 11.0 mg
Magnesium stearate: 5.0 mg
Labrasol™: 25.0 mg
Total weight of the tablet: 345 mg
Sildenafil citrate represents 14.5 wt % of the total weight of the tablet.

5 g of sildenafil citrate were placed in a mixer. 2.5 g of Labrasol were added under stirring in 5 aliquots of 0.5 g, after each addition the mixture was stirred for 5 minutes. After completion of Labrasol addition, 6.0 g of corn starch were added, and the mixture stirred for 5 minutes. 6.5 g of microcrystalline cellulose were added, and the mixture stirred 5 minutes. 75.0 g of Explotab™ were added, and the mixture stirred for 5 minutes. 15.0 g of PVP K25, 10.0 g of Compritol™, 3.0 g of menthol, and 10.0 g of aspartame were added, and the mixture stirred for 5 minutes. 6.0 g of talc, 11.0 g of Aerosil 15.0 g of Cutina HR and 5.0 g of magnesium stearate were added, and the mixture stirred for 5 minutes. The mixture was fed to a tablet press machine, and tablets according to the above composition were obtained.

The tablets prepared according to this procedure were tested on 41 volunteers, 15 Caucasian males of age comprised between 44 and 62 years and average weight of 78 kg, and 26 Asian males of age comprised between 30 and 50 years and average weight of 65 kg. During the test, efficacy, organoleptic response and tolerability were tested. The choice of two different ethnic groups is intended to verify that no significant difference exists in different ethnic groups in the response to the assumption of the tablets. Table 1 reports the results of the test.

TABLE 1

| | Evaluation | Evaluation | |
|---|---|---|---|
| | Caucasians | Asians | Notes |
| Dissolution | Good | Quick | 3 Cauc. and 5 Asians |
| | (about 3 | | report dissolution not |
| | minutes) | | complete, some small |
| | | | particles remained |
| Time before | 15-20′ | 10-15′ | |
| effect | after | | |
| | dissolution | | |
| Taste | Pleasant | Good | 10 Asians suggested |
| | (mint | | adding some more |
| | like) | | flavour |
| After | Good | Good | Cauc. with weight |
| 30′ | | | higher than 80 kg |
| | | | would have preferred |
| | | | 2 tablets, 2 Asians felt |
| | | | too stiffness |

1. Sublingual tablet comprising sildenafil citrate in an amount comprised between 10 mg and 150 mg, and a nonionic surfactant in an amount comprised between 20 and 100% by weight of the amount of sildenafil citrate, wherein the nonionic surfactant is a polyethoxylated compound comprising a polar and a non-polar part, wherein the polar part of the polyethoxylated compound comprises from 3 to 8 ethoxy groups and the non-polar part is a C₄-C₂₀ alkyl group or a C₇-C₂₀ aryl group.

2. The sublingual tablet according to claim 1, wherein sildenafil citrate is present in an amount comprised between 50 mg and 150 mg.

3. The sublingual tablet according to claim 1, wherein the non-polar part of the polyethoxylated compound is a C₆-C₁₂ alkyl group or a C₈-C₁₄ aryl group.

4. The sublingual tablet according to claim 1, wherein the tablet further comprises at least one of the following ingredients: sweeteners, fragrances, binders, lubricants, excipients.

5. The sublingual tablet according to claim 1, wherein the amount of nonionic surfactant is comprised between 30 and 80% by weight of the amount of sildenafil citrate.

6. The sublingual tablet according to claim 1, further comprising at least one disintegrant, preferably in an amount comprised between 100 and 200% by weight based on the amount of sildenafil citrate.

7. The sublingual tablet according to claim 6, comprising a disintegrant based on sodium starch glycolate.

8. The sublingual tablet according to claim 1, wherein sildenafil citrate is present in an amount equal to or higher than 10 wt % based on the total weight of the tablet.

9. Process for preparing a tablet comprising sildenafil citrate in an amount comprised between 10 mg and 150 mg, and a non-ionic surfactant in an amount comprised between 20 and 100% by weight of the amount of sildenafil citrate, wherein the nonionic surfactant is a polyethoxylated compound comprising a polar and a non-polar part, wherein the polar part of the polyethoxylated compound comprises from 3 to 8 ethoxy groups and the non-polar part is a C₄-C₂₀ alkyl group or a C₇-C₂₀ aryl group, which process comprises the steps of:
a. Introducing sildenafil citrate into a mixer;
b. Adding to the mixer containing sildenafil citrate the nonionic surfactant in at least three aliquots and mixing for 5 minutes after the addition of each aliquot.

10. The process of claim 9, further comprising the step of:
c. adding all the other ingredients one by one or in groups of two to four ingredients at the time and mixing for at least five minutes after each addition.

11. The process of claim 9, further comprising the step of:
d. feeding the mixture to a tablet press machine.
